(19) United States
(12) Patent Application Publication
(10) Pub. No.: US 2021/0369678 A1
(43) Pub. Date: Dec. 2, 2021

(54) BENZIMIDAZOLE DERIVATIVE FOR USE IN THE TREATMENT OF INFLAMMATORY DISORDERS

(71) Applicants: Akebia Therapeutics, Inc., Cambridge, MA (US); Janssen Pharmaceutica NV, Beerse (BE)

(72) Inventors: Michael Rabinowitz, Boston, MA (US); Hariharan Venkatesan, San Diego, CA (US); Mark D. Rosen, San Diego, CA (US)

(21) Appl. No.: 17/282,108

(22) PCT Filed: Oct. 2, 2019
(86) PCT No.: PCT/US19/54293

§ 371 Date: Apr. 1, 2021

Related U.S. Application Data
(60) Provisional application No. 62/740,748, filed on Oct. 3, 2018.

(51) Int. Cl. A61K 31/4184; A61K 45/06; A61P 1/04

(52) U.S. Cl. CPC A61K 31/4184; A61K 9/0053; A61P 1/04; A61K 45/06

(57) ABSTRACT

Provided herein are methods of treating and preventing inflammatory bowel disease in a patient. The methods provided herein comprise administering to the patient in need thereof an effective amount of a benzoimidazole compound, Compound 1, to treat or prevent an inflammatory bowel disease in a patient. Also provided are formulations and routes of administration of the compound.

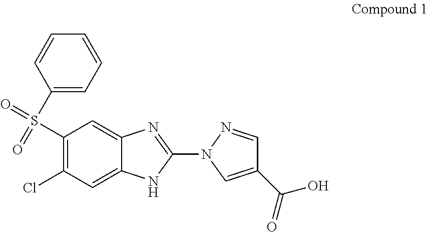 [SMILES: O=C(O)c1cnn(-c2nc3cc(S(=O)(=O)c4ccccc4)c(Cl)cc3[nH]2)c1]

### CROSS-REFERENCE TO RELATED APPLICATIONS

The present application claims benefit of U.S. Provisional Application No. 62/740,748, filed Oct. 3, 2018, which is hereby incorporated by reference in its entirety.

### INTRODUCTION

The present disclosure relates to a benzoimidazole compound, Compound 1, and pharmaceutically acceptable salts, tautomers, solvates, or hydrates thereof, to compositions comprising the compound, and to methods for treating or preventing inflammatory disorders, including inflammatory bowel diseases such as ulcerative colitis and Crohn's disease, using Compound 1.

### BACKGROUND

Inflammatory bowel disease (IBD) affects about 1.1 million people in the United States. IBD is an autoimmune disease marked by chronic inflammation of all or part of the gastrointestinal tract. The two major categories of IBD are ulcerative colitis and Crohn's disease. The symptoms of both ulcerative colitis and Crohn's disease include severe diarrhea, rectal bleeding, urgent need to move bowels, abdominal cramps, and pain, sensation of incomplete evacuation, constipation, fatigue, and weight loss. Ulcerative colitis affects the colon (large intestine) and rectum, and more specifically the inner lining (e.g., the epithelium) of the intestinal wall. Crohn's disease may affect any section of the gastrointestinal tract, including the mouth, esophagus, stomach, small intestine, large intestine, rectum, and anus. Crohn's disease may involve all layers of the intestinal wall.

Current therapies are directed to reducing the inflammatory process, and are often associated with adverse side effects, such as nausea, vomiting, anorexia, dyspepsia, malaise, headaches, abdominal pain, fever, rash, pancreatic, bone marrow suppression, formation of antibodies, infusion reactions, and increased opportunistic infections. Furthermore, 25-30% of ulcerative colitis patients ultimately need surgery. The available methods for treating inflammatory bowel disease (e.g., invasive surgical treatments) provide only treatment of symptoms rather than a substantial cure, and affect the patient's quality of life and may be life-threatening. Thus, there is a critical need in the art for compositions and methods that exhibit improved efficacy for the treatment of inflammatory bowel disease, including ulcerative colitis and Crohn's disease, while overcoming the safety concerns of existing therapeutic modalities, in particular, without inducing unwanted pharmacology, for example, when dosed orally.

International Patent Application Publication No. WO 2009/134750 is directed to various prolyl hydroxylase inhibitors. International Patent Application Publication No. WO 2009/134750 also provides that such compounds are useful in pharmaceutical compositions and methods for the treatment of disease states, disorders, and conditions modulated by prolyl hydroxylase activity.

### 3 SUMMARY OF THE INVENTION

In part, the present invention relates to the use of prolyl hydroxylase inhibitor compounds in IBD that do not induce unwanted pharmacology, particularly when dosed orally. Surprisingly, poorly absorbed compounds (e.g., Compound 1, or a pharmaceutically acceptable salt, tautomer, solvate, or hydrate thereof) can be therapeutically effective while reducing undesirable side effects (e.g., due to off-target effects and/or an undesired drug distribution profile).

In one embodiment, provided herein is a method for treating or preventing inflammatory bowel disease in a patient in need thereof, comprising administering orally to a patient in need thereof an effective amount of Compound 1:

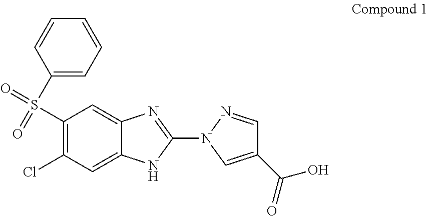 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In certain embodiments, the inflammatory bowel disease is ulcerative colitis. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis.

In certain embodiments, the patient has undergone prior treatment for ulcerative colitis. In certain embodiments, the patient is currently being treated with an anti-inflammatory drug. In certain embodiments, the inflammatory bowel disease is Crohn's disease. In certain embodiments, the Crohn's disease is ileocolitis. In certain embodiments, the Crohn's disease is ileitis. In certain embodiments, the Crohn's disease is gastroduodenal Crohn's disease. In certain embodiments, the Crohn's disease is jejunoileitis. In certain embodiments, the Crohn's disease is Crohn's (granulomatous) colitis. In certain embodiments, the patient has a Crohn's Disease Activity Index (“CDAI”) score greater than or equal to 150 and less than or equal to 220. In certain embodiments, the patient has a CDAI score greater than or equal to 220 and less than or equal to 300. In certain embodiments, the patient has a CDAI score greater than 300.

In certain embodiments, the oral bioavailability of Compound 1 is less than 10%. In certain embodiments, the compound is delivered to the intestinal epithelium of the patient with negligible systemic absorption. In certain embodiments, the compound is delivered to the colon of the patient with negligible systemic absorption. In certain embodiments, the compound is delivered to the lining of the descending colon of the patient with negligible systemic absorption. In certain embodiments, serum levels of the compound in the patient are negligible at a time after administration of the compound. In certain embodiments, the change in hemoglobin levels in the patient are negligible six-hours after administration of the compound. In certain embodiments, the change in EPO levels in the patient are negligible six-hours after oral administration of the compound. In certain embodiments, the change in red blood cell levels in the patient are negligible six-hours after oral administration of the compound.

In certain embodiments, the patient is at risk of developing a cardiovascular disease. In certain embodiments, the patient has experienced a cardiovascular event in the last two years. In certain embodiments, the cardiovascular event is a heart attack or stroke. In certain embodiments, the patient has a disorder in which angiogenesis is contraindicated. In certain embodiments, the patient has a disorder in which erythropoiesis is contraindicated.

In certain embodiments, the composition is administered daily. In certain embodiments, Compound 1 is administered as an oral formulation. In certain embodiments, the oral formulation is a tablet or capsule. In certain embodiments, the oral formulation does not comprise an enteric coating.

In one embodiment, provided herein is a method of healing the epithelial layer of the descending colon in a patient in need thereof by administering orally an effective amount of Compound 1:

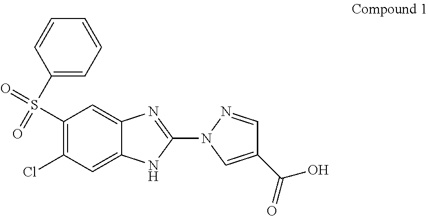 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In certain embodiments, the bioavailability of Compound 1 is less than 10%. In certain embodiments, the compound is delivered to the intestinal epithelium of the patient with negligible systemic absorption. In certain embodiments, the compound is delivered to the colon of the patient with negligible systemic absorption. In certain embodiments, the compound is delivered to the lining of the descending colon of the patient with negligible systemic absorption. In certain embodiments, serum levels of the compound in the patient are negligible at a time after administration of the compound. In certain embodiments, the change in hemoglobin levels in the patient are negligible six-hours after administration of the compound. In certain embodiments, the change in EPO levels in the patient are negligible six-hours after oral administration of the compound. In certain embodiments, the change in red blood cell levels in the patient are negligible six-hours after oral administration of the compound.

In certain embodiments, the patient is at risk of developing a cardiovascular disease. In certain embodiments, the patient has a disorder in which angiogenesis is contraindicated. In certain embodiments, the patient has a disorder in which erythropoiesis is contraindicated.

In certain embodiments, the composition is administered daily. In certain embodiments, the composition is formulated as an oral formulation. In certain embodiments, the oral formulation is a tablet or capsule. In certain embodiments, the oral formulation does not comprise an enteric coating.

In one embodiment, provided herein is a method for maintaining remission of ulcerative colitis in a patient, comprising administering orally to a patient an effective amount of Compound 1:

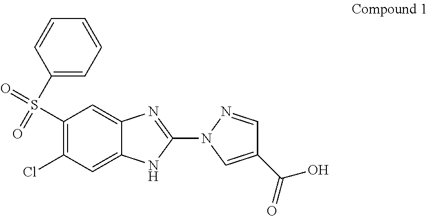 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof, after remission has been achieved. In certain embodiments, the ulcerative colitis is mild to moderate ulcerative colitis. In certain embodiments, the ulcerative colitis is moderate to severe ulcerative colitis. In certain embodiments, the remission has been achieved by treatment with immunosuppressive agents, inflammatory agents, or surgery.

In one embodiment, provided herein is a method for inducing remission of ulcerative colitis or Crohn's disease in a patient, comprising administering orally to a patient an effective amount of Compound 1:

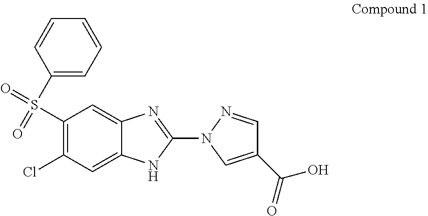 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof after remission has been achieved. In certain embodiments, the ulcerative colitis is mild to moderate ulcerative colitis. In certain embodiments, the ulcerative colitis is moderate to severe ulcerative colitis.

In one embodiment, provided herein is a method for inducing mucosal healing in a patient in need thereof, comprising administering orally to a patient an effective amount of Compound 1:

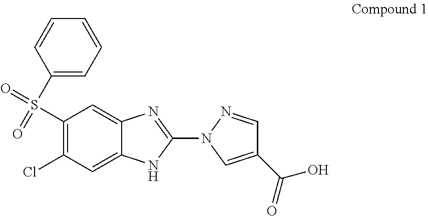 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In one embodiment, provided herein is a method for treating or preventing ulcerative colitis in a male patient, comprising administering orally to a male patient an effective amount of Compound 1:

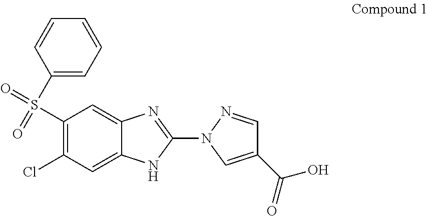 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof, wherein the hemoglobin level in the male patient is 13.5 to 17.5 grams per deciliter. In certain embodiments, the ulcerative colitis is mild to moderate ulcerative colitis. In certain embodiments, the ulcerative colitis is moderate to severe ulcerative colitis.

In one embodiment, provided herein is a method for treating or preventing ulcerative colitis in a female patient, comprising administering orally to a female patient an effective amount of Compound 1:

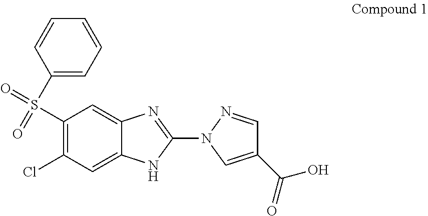 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof, wherein the hemoglobin level in the female patient is 12.0 to 15.5 grams per deciliter. In certain embodiments, the ulcerative colitis is mild to moderate ulcerative colitis. In certain embodiments, the ulcerative colitis is moderate to severe ulcerative colitis.

In one embodiment, provided herein is a method for treating or preventing esophagitis in a patient in need thereof, comprising administering to a patient in need thereof an effective amount of Compound 1:

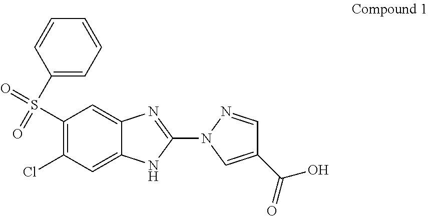 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof. In certain embodiments, the esophagitis is eosinophilic esophagitis.

In one embodiment, provided herein is a method for treating or preventing gastritis in a patient in need thereof, comprising administering to a patient in need thereof an effective amount of Compound 1:

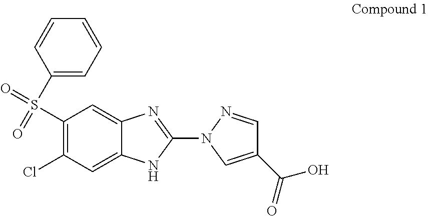 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In one embodiment, provided herein is a method for treating or preventing pouchitis in a patient in need thereof, comprising administering to a patient in need thereof an effective amount of Compound 1:

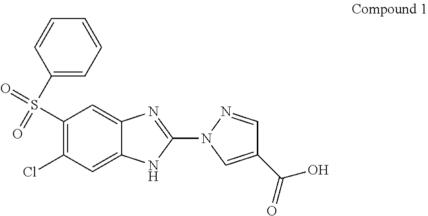 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In one embodiment, provided herein is a method for treating or preventing mucositis in a patient in need thereof, comprising administering to a patient in need thereof an effective amount of Compound 1:

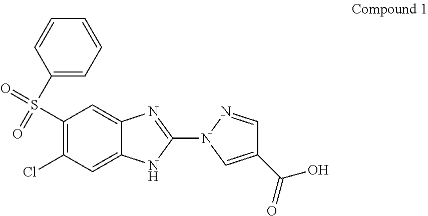 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In one embodiment, provided herein is an oral formulation comprising
(i) Compound 1:

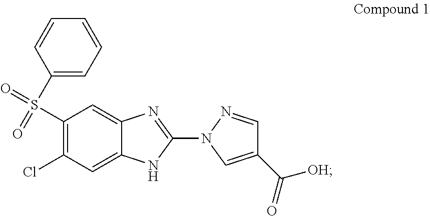 [SMILES: O=C(O)c1cnn(-c2nc3cc(S(=O)(=O)c4ccccc4)c(Cl)cc3[nH]2)c1]

or a pharmaceutically acceptable salt, tautomer, solvate or hydrate thereof, and
(ii) one or more excipients or carriers suitable for oral administration. In certain embodiments, the oral formulation is a tablet or capsule. In certain embodiments, the oral formulation does not comprise an enteric coating.

### BRIEF DESCRIPTION OF THE DRAWINGS

FIG. 1 depicts the EPO response to oral (p.o.) and intraperitoneal (i.p.) doses of Compound 1 at 6 h in BalbC mice.

FIG. 2 depicts the plasma concentration of Compound 1 in healthy male SD rats over 24 hours.

FIG. 3 depicts the serum hemoglobin concentration after 14 days of oral dosing Compound 1 in mice with dextran sulfate sodium (DSS)-induced colitis.

FIG. 4 depicts Endoscopy Colitis Scores in mice with dextran sulfate sodium (DSS)-induced colitis dosed via oral gavage with Compound 1, 6-thioguanine, or vehicle control once a day after 19 days.

FIG. 5 depicts the study design of the TNBS (2,4,6-trinitrobenzene sulfonic acid) colitis model.

FIG. 6 depicts the effect of Compound 1 on body weight loss in TNBS colitis model and illustrates that Compound 1 protects against body weight loss in the TNBS model.

FIG. 7 depicts the effect of Compound 1 on colon weight/length ratio in TNBS colitis model and illustrates that Compound 1 improves colon weight/length ratio in the TNBS model.

FIG. 8 depicts the histopathology inflammation score for Compound 1 in TNBS colitis model and illustrates that Compound 1 improves the histopathology inflammation score in the TNBS model.

FIG. 9 depicts the histopathology summed score for Compound 1 in TNBS colitis model and illustrates that Compound 1 improves the histopathology summed score in the TNBS model.

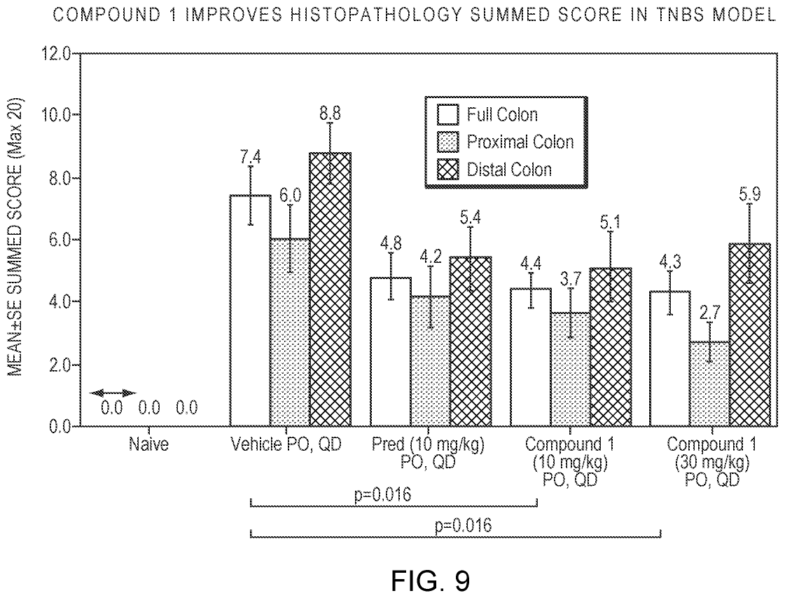

FIG. 10 depicts the mean (±SD) plasma concentration of Compound 1 in healthy male beagle dogs over 8 hours following intravenous and oral dosing of Compound 1.

FIG. 11 depicts the mean (±SD) plasma concentration of Compound 1 in healthy male cynomolgus monkeys over 24 hours following intravenous and oral dosing of Compound 1.

### DETAILED DESCRIPTION OF THE METHODS

### Overview

Hypoxia-Inducible Factor (HIF) prolyl hydroxylase inhibitors (PHDi) have been reported to be effective for treating anemia due to their ability to stimulate erythropoiesis, leading to increased erythropoietin (EPO) and hematocrit levels (see, e.g., Ariazi et al., Discovery and preclinical characterization of GSK1278863 (Daprodustat), a small molecule hypoxia inducible factor-prolyl hydroxylase inhibitor for anemia, The Journal of Pharmacology and Experimental Therapeutics 363:336-47, December 2017; Debenham et al., Discovery of N-[Bis(4-methoxyphenyl)methyl]-4-hydroxy-2-(pyridazine-3-yl)pyrimidine-5-carboxamide (MK-8617), an orally active pan-inhibitor of hypoxia-inducible factor prolyl hydroxylase 1-3 (HIF PHDI-3) for the treatment of anemia, J. Med. Chem. 2016, 59, 11039-49; and Beck et al., Discovery of Molidustat (BAY 85-3934): a small-molecule oral HIF-prolyl hydroxylase (HIF-PH) inhibitor for the treatment of renal anemia, ChemMedChem 2018, 13, 988-1003). However, for inflammatory bowel disease (IBD), systemic effects, including increases in EPO and hematocrit, are undesirable and may impede efficacious dosing.

Provided herein Compound 1 was dosed orally (p.o.) and showed excellent efficacy in both mouse models of inflammatory bowel disease. Unexpectedly, the excellent efficacy was observed even though Compound 1 has poor absorption in low (<5%) systemic bioavailability across preclinical species (rat, dog, and monkey) following p.o. dosing and no systemic pharmacology resulting in increases in EPO/hematocrit was observed. Accordingly, the present invention relates to uses of a prolyl hydroxylase inhibitor compound (e.g., Compound 1) in IBD that do not induce unwanted pharmacology, particularly when dosed orally.

Also provided herein are methods of treating and preventing inflammatory bowel disease in a patient. The methods provided herein comprise administering to the patient in need thereof an effective amount of Compound 1, a benzoimidazole compound, to treat or prevent an inflammatory bowel disease in a patient. Specifically, provided herein is a method for treating and or preventing an IBD, such as Crohn's disease or ulcerative colitis, in a patient wherein the method comprises administering orally to the patient a therapeutically effective dose of Compound 1 without significantly increasing hemoglobin levels or red blood cell levels of the patient. General methods of treatment and prevention are described in Section 5.4. Combination therapies using the compound described herein are described in Section 5.6. Formulations and routes of administration of the compound described herein are described in Section 5.7.

### Definitions

In certain embodiments, as used throughout the description and claims of this specification the word “comprise” and other forms of the word, such as “comprising” and “comprises,” means including but not limited to, and is not intended to exclude, for example, other additives, components, integers, or steps. In certain embodiments, as used in the description and the appended claims, the singular forms “a,” “an,” and “the” include plural referents unless the context clearly dictates otherwise. Thus, for example, reference to “a composition” includes mixtures of two or more such compositions. In certain embodiments, “optional” or “optionally” means that the subsequently described event or circumstance can or cannot occur, and that the description includes instances where the event or circumstance occurs and instances where it does not.

As used herein, an “effective amount” refers to that amount of a compound or a pharmaceutically acceptable salt, tautomer, solvate, or hydrate thereof, sufficient to provide a therapeutic benefit in the treatment of the disease or to delay or minimize symptoms associated with the disease.

As used herein, the terms “treat,” “treating,” and “treatment” refer to the reversing, ameliorating, reducing, or arresting a symptom, clinical sign, and/or underlying pathology of a condition or disease.

As used herein, the terms “prevent,” “preventing,” and “prevention” refer to reducing the frequency of, decreasing the severity of, reducing the recurrence of, or delaying the onset of, a symptom of a medical condition in a subject.

As used herein, the term “pharmaceutically acceptable salt” refers to a salt prepared from pharmaceutically acceptable non-toxic acids or bases including inorganic acids and bases and organic acids and bases. Suitable pharmaceutically acceptable salts are potassium salts and hydrochloride salts.

In certain embodiments, “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

As used herein, the term “hydrate” means a compound provided herein or a pharmaceutically acceptable salt thereof, that further includes a stoichiometric or non-stoichiometric amount of water bound by non-covalent intermolecular forces.

As used herein, the term “solvate” means a compound provided herein or a pharmaceutically acceptable salt thereof, that further includes a stoichiometric or non-stoichiometric amount of a solvent, other than water, bound by non-covalent intermolecular forces.

“Tautomer” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

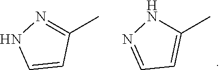

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range. In certain embodiments, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In certain embodiments, the term subject or patient can refer to a mammal, such as a human, mouse, dog, donkey, horse, rat, guinea pig, bird, or monkey. In specific embodiments, a subject or a patient is a human subject or patient.

In certain embodiments, Compound 1 can be used with the methods and compositions provided herein. Compound 1 is 1-(6-chloro-5-(phenylsulfonyl)-1H-benzo[d]imidazol-2-yl)-1H-pyrazole-4-carboxylic acid, having the structure:

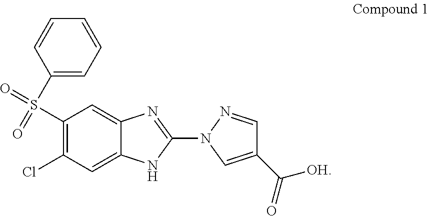 [SMILES: O=C(O)c1cnn(-c2nc3cc(S(=O)(=O)c4ccccc4)c(Cl)cc3[nH]2)c1]

In certain embodiments, a pharmaceutically acceptable salt of Compound 1 can be used with the methods or compositions provided herein. In certain embodiments, a tautomer of Compound 1 can be used with the methods or compositions provided herein. An example of a tautomer of Compound 1 is as follows:

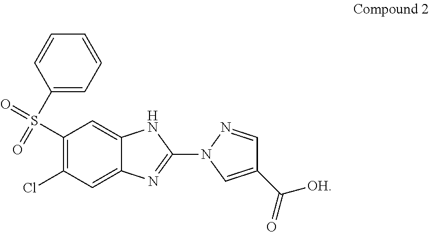 [SMILES: O=C(O)c1cnn(-c2nc3cc(Cl)c(S(=O)(=O)c4ccccc4)cc3[nH]2)c1]

In certain embodiments, a solvate of Compound 1 can be used with the methods or compositions provided herein. In certain embodiments, a hydrate of Compound 1 can be used with the methods or compositions provided herein.

As used herein, the term “HIF prolyl hydroxylase” is art-recognized and may be abbreviated as “PHD”. HIF prolyl hydroxylase is also known as “prolyl hydroxylase domain-containing protein” which may be abbreviated as “PHD”. In this regard, there are three different PHD isoforms, PHD1, PHD2, and PHD3, also referred to as EGLN2, EGLN1, and EGLN3, or HPH3, HPH2, and HPH1, respectively. In certain embodiments, HIF prolyl hydroxylase may refer to a particular target of the enzyme (e. g. , HIF-la prolyl hydroxylase, HIF-2a prolyl hydroxylase, and/or HIF-3a prolyl hydroxylase).

### Compounds

In certain embodiments, a compound for use with the methods provided herein is a benzoimidazole compound. In certain embodiments, the benzoimidazole compound is 1-(6-chloro-5-(phenylsulfonyl)-1H-benzo[d]imidazol-2-yl)-1H-pyrazole-4-carboxylic acid (Compound 1):

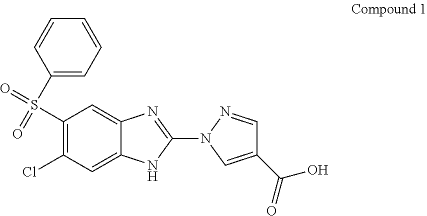 [SMILES: O=C(O)c1cnn(-c2nc3cc(S(=O)(=O)c4ccccc4)c(Cl)cc3[nH]2)c1]

or a pharmaceutically acceptable salt, tautomer, solvate, or hydrate thereof.

In certain embodiments, a compound for use with the methods provided herein is a benzoimidazole compound. In certain embodiments, the benzoimidazole compound is 1-(5-chloro-6-(phenylsulfonyl)-1H-benzo[d]imidazol-2-yl)-1H-pyrazole-4-carboxylic acid (Compound 2):

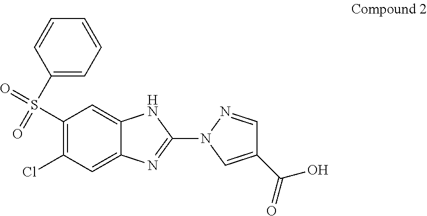 [SMILES: O=C(O)c1cnn(-c2nc3cc(Cl)c(S(=O)(=O)c4ccccc4)cc3[nH]2)c1]

or a pharmaceutically acceptable salt, tautomer, solvate, or hydrate thereof.

In certain embodiments, provided herein is a mixture of Compound 1 and Compound 2 for use with the methods provided herein. While the detailed description is drafted with a focus on Compound 1, unless otherwise indicated, Compound 2 or a mix of Compound 1 and Compound 2 can be put in place of Compound 1.

Compound 1 can be prepared using reagents and methods known in the art, including the methods provided in International Patent Application Publication No. WO 2009/134750, which is incorporated herein by reference in its entirety.

Without being bound by theory, in certain embodiments, Compound 1 and pharmaceutically acceptable salts, tautomers, solvates, or hydrates thereof for use with the methods provided herein are modulators of a HIF prolyl hydroxylase. In more specific embodiments, Compound 1 and pharmaceutically acceptable salts, tautomers, solvates, or hydrates thereof for use with the methods provided herein are modulators of a HIF-1-α prolyl hydroxylase. In other, more specific embodiments, Compound 1 and pharmaceutically acceptable salts, tautomers, solvates, or hydrates thereof for use with the methods provided herein are modulators of a HIF-2-α prolyl hydroxylase. In certain embodiments, Compound 1 and pharmaceutically acceptable salts, tautomers, solvates, or hydrates thereof for use with the methods provided herein are stabilizers of HIF.

It should be noted that if there is a discrepancy between a depicted structure and a name given that structure, the depicted structure is to be accorded more weight.

### Methods of Use

Section 5.4.1 provides methods of treatment and prevention of inflammatory bowel diseases, as well as methods of healing the mucosal and epithelial layers of a patient in need thereof. Section 5.4.2 discloses tissue specific effects of Compound 1, as well as the oral bioavailability and absorption properties of Compound 1. Section 5.4.3 discloses specific patient populations to be treated with the methods described herein.

Methods described herein can induce the desired therapeutic effect while also not inducing unwanted pharmacology, particularly when dosed orally. That is, poorly absorbed compounds such as Compound 1, or a pharmaceutically acceptable salt, tautomer, solvate, or hydrate thereof, can be therapeutically effective while reducing undesirable side effects (e.g., due to off-target effects and/or an undesired drug distribution profile).

### Methods of Treatment and Prevention

The methods provided herein can be used to efficaciously administer to a patient a benzoimidazole compound to treat and/or prevent an inflammatory bowel disease. Such inflammatory bowel diseases include, but are not limited to, ulcerative colitis and Crohn's disease. In specific embodiments, the benzoimidazole compound is administered orally to a patient.

In certain embodiments provided herein is a method for treating or preventing an inflammatory bowel disease, comprising administering to a patient having an inflammatory bowel disease a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable salt, tautomer, solvate, or hydrate thereof are administered. In certain embodiments, the inflammatory bowel disease is ulcerative colitis. In certain embodiments, the inflammatory bowel disease is Crohn's disease. In certain embodiments, the treating or preventing comprises decreasing the frequency and severity of flare-ups associated with the inflammatory bowel disease.

In certain embodiments provided herein is a method for treating or preventing ulcerative colitis, comprising administering to a patient having ulcerative colitis a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable salt, tautomer, solvate, or hydrate thereof are administered. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis. In certain embodiments, the treating or preventing comprises decreasing the frequency and severity of flare-ups associated with the ulcerative colitis.

In certain embodiments provided herein is a method for treating or preventing Crohn's disease, comprising administering to a patient having Crohn's disease a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable salt, tautomer, solvate, or hydrate thereof are administered. In certain embodiments, the Crohn's disease is ileocolitis. In certain embodiments, the Crohn's disease is ileitis. In certain embodiments, the Crohn's disease is gastroduodenal Crohn's disease. In certain embodiments, the patient has a Crohn's Disease Activity Index (“CDAI”) score greater than 140, 150, 160, 170, 180, 190, 200, 210, 220, 230, 240, 250, 260, 270, 280, 290, or 300. In certain embodiments, the patient has a CDAI score less than 150, 160, 170, 180, 190, 200, 210, 220, 230, 240, 250, 260, 270, 280, 290, or 300. In certain embodiments, the patient has a CDAI score greater than or equal to 150 and less than or equal to 220. In certain embodiments, the patient has a CDAI score greater than or equal to 220 and less than or equal to 300. In certain embodiments, the treating or preventing comprises decreasing the frequency and severity of flare-ups associated with the Crohn's disease.

In certain embodiments provided herein is a method for reducing or alleviating a symptom of an inflammatory bowel disease in a patient in need thereof, comprising administering to a patient having an inflammatory bowel disease a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable tautomer, salt, tautomer, solvate, or hydrate thereof are administered. In certain embodiments, the inflammatory bowel disease is ulcerative colitis. In certain embodiments, the inflammatory bowel disease is Crohn's disease. In certain embodiments, the symptom is selected from one of the following: severe diarrhea, rectal bleeding, urgent need to move bowels, abdominal cramps and pain, sensation of incomplete evacuation, constipation, fatigue, and weight loss. In certain embodiments, the section of the colon is the ileum. In a more specific embodiment, a sufficient number of successive doses of Compound 1, or a pharmaceutically acceptable salt, tautomer, solvate, or hydrate thereof, are administered. In certain embodiments, the composition is administered orally.

In certain embodiments provided herein is a method for maintaining remission of ulcerative colitis or Crohn's disease in a patient, wherein the method comprises administering to the patient a composition comprising an effective amount of Compound 1, or a pharmaceutically acceptable salt, tautomer, solvate, or hydrate thereof, after the remission has been achieved. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis. In certain embodiments, the Crohn's disease is ileocolitis. In certain embodiments, the Crohn's disease is ileitis. In certain embodiments, the Crohn's disease is gastroduodenal Crohn's disease. In certain embodiments, the remission has been achieved by treatment with immunosuppressive agents, inflammatory agents, or surgery. In certain embodiments, the immunosuppressive agent is a glucocorticoid, azathioprine, infliximab, adalimumab, golimumab, methotrexate, natalizumab, ustekinumab, mercaptopurine, dactinomycin, anthracycline, mitomycin C, bleomycin, mithramycin, tacrolimus, cyclosporine, sirolimus, everolimus, an opioid, an interferon, a TNF binding protein, mycophenolate, fingolimod, or myriocin. In certain embodiments, the anti-inflammatory agent is sulfasalazine, an aminosalicylate, a corticosteroid, aspirin, ibuprofen, naproxen, paracetamol, celecoxib, diclofenac, diflunisal, etodolac, indomethacin, ketoprofen, ketorolac, nabumetone, oxaprozin, piroxicam, salsalate, sulindac, or tolmetin.

In certain embodiments provided herein is a method for inducing remission of ulcerative colitis or Crohn's disease in a patient, wherein the method comprises administering to the patient a composition comprising an effective amount of Compound 1, or a pharmaceutically acceptable salt, tautomer, solvate, or hydrate thereof. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis. In certain embodiments, the Crohn's disease is ileocolitis. In certain embodiments, the Crohn's disease is ileitis. In certain embodiments, the Crohn's disease is gastroduodenal Crohn's disease. In certain embodiments, the composition is administered orally.

In certain embodiments provided herein is a method for inducing mucosal healing in a patient in need thereof, wherein the method comprises orally administering to the patient a composition comprising an effective amount of Compound 1, or a pharmaceutically acceptable salt, tautomer, solvate, or hydrate thereof. In certain embodiments, the patient does not have an inflammatory bowel disease. In certain embodiments, the patient does not have ulcerative colitis. In certain embodiments, the patient does not have Crohn's disease. In certain embodiments, the patient has an inflammatory bowel disease. In certain embodiments, the inflammatory bowel disease is ulcerative colitis. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis. In certain embodiments, the inflammatory bowel disease is Crohn's disease. In certain embodiments, the Crohn's disease is ileocolitis. In certain embodiments, the Crohn's disease is ileitis. In certain embodiments, the Crohn's disease is gastroduodenal Crohn's disease. In certain embodiments, the patient has a Crohn's Disease Activity Index (“CDAI”) score greater than 140, 150, 160, 170, 180, 190, 200, 210, 220, 230, 240, 250, 260, 270, 280, 290, or 300. In certain embodiments, the patient has a CDAI score less than 150, 160, 170, 180, 190, 200, 210, 220, 230, 240, 250, 260, 270, 280, 290, or 300. In certain embodiments, the patient has a CDAI score greater than or equal to 150 and less than or equal to 220. In certain embodiments, the patient has a CDAI score greater than or equal to 220 and less than or equal to 300.

In certain embodiments provided herein is a method for treating or preventing ulcerative colitis in a male patient, comprising administering to the male patient a composition comprising an effective amount of Compound 1, or a pharmaceutically acceptable salt, tautomer, solvate, or hydrate thereof. In certain embodiments, the hemoglobin level in the male patient is 13.5 to 17.5 grams per deciliter. In a more specific embodiment, a sufficient number of successive doses of Compound 1, or a pharmaceutically acceptable salt, tautomer, solvate or hydrate thereof are administered. In certain embodiments, the composition is administered orally. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis.

In certain embodiments provided herein is a method for treating or preventing ulcerative colitis in a female patient, comprising administering to the female patient a composition comprising an effective amount of Compound 1, or a pharmaceutically acceptable salt, tautomer, solvate, or hydrate thereof. In certain embodiments, the hemoglobin level in the female patient is 12.0 to 15.5 grams per deciliter. In certain embodiments, the composition is administered orally. In a more specific embodiment, a sufficient number of successive doses of Compound 1, or a pharmaceutically acceptable salt, tautomer, solvate, or hydrate thereof, are administered. In certain embodiments, the ulcerative colitis is mild to moderate ulcerative colitis. In certain embodiments, the ulcerative colitis is moderate to severe ulcerative colitis. In certain embodiments, the ulcerative colitis is ulcerative proctitis. In certain embodiments, the ulcerative colitis is proctosigmoiditis. In certain embodiments, the ulcerative colitis is left-sided colitis. In certain embodiments, the ulcerative colitis is pan-ulcerative colitis.

In certain embodiments provided herein is a method of healing the epithelial layer of the colon or a section of the colon in a patient in need thereof, comprising oral administration to a patient in need thereof a composition comprising an effective amount of Compound 1, or a pharmaceutically acceptable salt, tautomer, solvate, or hydrate thereof. In certain embodiments, the section of the colon is the ascending colon. In certain embodiments, the section of the colon is the transverse colon. In certain embodiments, the area of the colon is the descending colon. In certain embodiments, the area of the colon is the sigmoid colon. In certain embodiments, the area of the colon is the rectum. In a more specific embodiment, a sufficient number of successive doses of Compound 1, or a pharmaceutically acceptable salt, tautomer, solvate, or hydrate thereof, are administered.

In certain embodiments, provided herein is a method of healing the epithelial layer of the small intestine or a section of the small intestine in a patient in need thereof, comprising oral administration to a patient in need thereof a composition comprising an effective amount of Compound 1, or a pharmaceutically acceptable salt, tautomer, solvate, or hydrate thereof. In certain embodiments, the section of the small intestine is the duodenum. In certain embodiments, the section of the small intestine is the jejunum. In certain embodiments, the section of the colon is the ileum. In a more specific embodiment, a sufficient number of successive doses of Compound 1, or a pharmaceutically acceptable salt, tautomer, solvate, or hydrate thereof, are administered.

In certain embodiments provided herein is a method for treating or preventing esophagitis in a patient in need thereof, comprising administering to the patient a composition comprising an effective amount of Compound 1, or a pharmaceutically acceptable salt, tautomer, solvate or hydrate thereof. In a more specific embodiment, a sufficient number of successive doses of Compound 1, or a pharmaceutically acceptable salt, tautomer, solvate, or hydrate thereof, are administered. In certain embodiments, the esophagitis is eosinophilic esophagitis. In certain embodiments, the treating or preventing comprises decreasing the frequency and severity of flare-ups associated with the esophagitis. In certain embodiments, the administering is performed orally.

In certain embodiments, provided herein is a method for treating or preventing gastritis in a patient in need thereof, comprising administering to the patient a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable salt, tautomer, solvate, or hydrate thereof, are administered. In certain embodiments, the treating or preventing comprises decreasing the frequency and severity of flare-ups associated with the gastritis. In certain embodiments, the administering is performed orally.

In certain embodiments, provided herein is a method for treating or preventing pouchitis in a patient in need thereof, comprising administering to the patient a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable salt, tautomer, solvate or hydrate thereof are administered. In certain embodiments, the treating or preventing comprises decreasing the frequency and severity of flare-ups associated with the pouchitis. In certain embodiments, the administering is performed orally.

In certain embodiments, provided herein is a method for treating or preventing mucositis in a patient in need thereof, comprising administering to the patient a composition comprising an effective amount of Compound 1, or a pharmaceutically acceptable salt, tautomer, solvate, or hydrate thereof. In a more specific embodiment, a sufficient number of successive doses of Compound 1, or a pharmaceutically acceptable salt, tautomer, solvate, or hydrate thereof, are administered. In certain embodiments, the treating or preventing comprises decreasing the frequency and severity of flare-ups associated with the mucositis. In certain embodiments, the administering is performed orally.

### Tissue Specific Effects of Compound 1

In certain embodiments, the erythropoietin (“EPO”) levels of the patient are measured after oral administration of Compound 1. In certain embodiments, Compound 1 does not increase the EPO levels of the patient at a time following oral administration of Compound 1. In certain embodiments, the change in EPO levels in the patient are measured immediately, 15 minutes, 30 minutes, 1 hour, 2 hours, 4 hours, 6 hours, 8 hours, 12 hours, 16 hours, 24 hours, 2 days, 3 days, 4 days 5 days, 6 days, 1 week, or 2 weeks following oral administration of Compound 1. In certain embodiments, the change in EPO levels in the patient are negligible immediately, 15 minutes, 30 minutes, 1 hour, 2 hours, 4 hours, 6 hours, 8 hours, 12 hours, 16 hours, 24 hours, 2 days, 3 days, 4 days, 5 days, 6 days, 1 week, or 2 weeks following oral administration of Compound 1. In certain embodiments, the change in EPO level following administration of Compound 1 is at most 20%, 15%, 10%, 9%, 8%, 7%, 6%, 5%, 4%, 3%, 2%, 1%, or 0.1% of the EPO level in the same patient prior to administration of Compound 1. In certain embodiments, the change in EPO level following administration of Compound 1 is at most 20%, 15%, 10%, 9%, 8%, 7%, 6%, 5%, 4%, 3%, 2%, 1%, or 0.1% of the EPO level in an average healthy individual of the same gender and age range as the patient. In certain embodiments, the average EPO level in a healthy individual is between 4 and 24 milliunits per milliliter. In certain embodiments, the average EPO level in a healthy man is between 5.8 and 9.9 milliunits per milliliter. In certain embodiments, the average EPO level in a healthy woman is between 6.0 and 10.6 milliunits per milliliter. In certain embodiments, the change in EPO level following administration of Compound 1 is at most 6, 5.5, 5.0, 4.5, 4.0, 3.5, 3.0, 2.5, 2.0, 1.5, 1.0, 0.5, 0.1, or 0.0 millunits per milliliter more than in the same patient prior to administration of Compound 1.

In certain embodiments, the hemoglobin levels of the patient are measured after oral administration of Compound 1. In certain embodiments, Compound 1 does not increase the hemoglobin levels of the patient at a time following oral administration of Compound 1. In certain embodiments, the change in hemoglobin levels in the patient are measured immediately, 15 minutes, 30 minutes, 1 hour, 2 hours, 4 hours, 6 hours, 8 hours, 12 hours, 16 hours, 24 hours, 2 days, 3 days, 4 days 5 days, 6 days, 1 week, or 2 weeks following oral administration of Compound 1. In certain embodiments, the change in hemoglobin levels in the patient are negligible after immediately, 15 minutes, 30 minutes, 1 hour, 2 hours, 4 hours, 6 hours, 8 hours, 12 hours, 16 hours, 24 hours, 2 days, 3 days, 4 days, 5 days, 6 days, 1 week or 2 weeks following oral administration of Compound 1. In certain embodiments, the change in hemoglobin level following administration of Compound 1 is at most 20%, 15%, 10%, 9%, 8%, 7%, 6%, 5%, 4%, 3%, 2%, 1%, or 0.1% of the hemoglobin level in the same patient prior to administration of Compound 1. In certain embodiments, the change in hemoglobin level following administration of Compound 1 is at most 20%, 15%, 10%, 9%, 8%, 7%, 6%, 5%, 4%, 3%, 2%, 1%, or 0.1% of the hemoglobin level in an average healthy individual of the same gender and age range as the patient. In certain embodiments, the average hemoglobin level in a healthy man is between 13.5 to 17.5 grams per deciliter. In certain embodiments, the average hemoglobin level in a healthy woman is between 12.0 and 15.5 grams per deciliter. In certain embodiments, the change in hemoglobin level following administration of Compound 1 is at most 5.0, 4.5, 4.0, 3.5, 3.0, 2.5, 2.0, 1.5, 1.0, 0.5, 0.1, or 0.0 grams per deciliter more than in the same patient prior to administration of Compound 1.

In certain embodiments, the red blood cell levels of the patient are measured after oral administration of Compound 1. In certain embodiments, Compound 1 does not increase the red blood cell levels of the patient at a time following oral administration of Compound 1. In certain embodiments, the change in red blood cell levels in the patient are measured immediately, 15 minutes, 30 minutes, 1 hour, 2 hours, 4 hours, 6 hours, 8 hours, 12 hours, 16 hours, 24 hours, 2 days, 3 days, 4 days 5 days, 6 days, 1 week, or 2 weeks following oral administration of Compound 1. In certain embodiments, the change in red blood cell levels following administration of Compound 1 is at most 20%, 15%, 10%, 9%, 8%, 7%, 6%, 5%, 4%, 3%, 2%, 1%, or 0.1% of the red blood cell levels in the same patient prior to administration of Compound 1. In certain embodiments, the change in red blood cell levels following administration of Compound 1 is at most 20%, 15%, 10%, 9%, 8%, 7%, 6%, 5%, 4%, 3%, 2%, 1%, or 0.1% of the red blood cell levels in an average healthy individual of the same gender and age range as the patient. In certain embodiments, the average red blood cell levels in a healthy man are between 4.7 to 6.1 million cells/μL. In certain embodiments, the average red blood cell levels in a healthy woman are between 4.2 to 5.4 million cells/μL. In certain embodiments, the change in red blood cell levels following administration of Compound 1 is at most 1.5, 1.4, 1.3, 1.2, 1.1, 1.0, 0.9, 0.8, 0.7, 0.6, 0.5, 0.4, 0.3, 0.2, 0.1, 0.05, 0.01, 0.005 million cells/μL more than in the same patient prior to administration of Compound 1.

In certain embodiments, the oral bioavailability of Compound 1 is limited. In certain embodiments, the oral bioavailability of Compound 1 is less than 10%. In certain embodiments, the oral bioavailability of Compound 1 is less than 9%. In certain embodiments, the oral bioavailability of Compound 1 is less than 8%. In certain embodiments, the oral bioavailability of Compound 1 is less than 7%. In certain embodiments, the oral bioavailability of Compound 1 is less than 6%. In certain embodiments, the oral bioavailability of Compound 1 is less than 5%. In certain embodiments, the oral bioavailability of Compound 1 is less than 4%. In certain embodiments, the oral bioavailability of Compound 1 is less than 3%. In certain embodiments, the oral bioavailability of Compound 1 is less than 2%. In certain embodiments, the oral bioavailability of Compound 1 is less than 1%. In certain embodiments, the oral bioavailability of Compound 1 is less than 0.5%. In certain embodiments, the oral bioavailability of Compound 1 is less than 0.1%. In certain embodiments, when Compound 1 is administered orally it is absorbed at minimal amounts. In certain embodiments, the amount of systemic absorption of Compound 1 is negligible. In certain embodiments, at most 40%, 30%, 25%, 20%, 15%, 10%, 9%, 8%, 7%, 6%, 5%, 4%, 3%, 2%, 1%, 0.5%, 0.1%, 0.05%, or 0.01% of the orally administered Compound 1 by weight is detectable in the serum.

In certain embodiments, Compound 1 is delivered to the intestinal epithelium with negligible systemic absorption. In certain embodiments, Compound 1 is delivered to the colon with negligible systemic absorption. In certain embodiments, Compound 1 is delivered to the lining of the ascending colon with negligible systemic absorption. In certain embodiments, Compound 1 is delivered to the lining of the transverse colon with negligible systemic absorption. In certain embodiments, Compound 1 is delivered to the lining of the descending colon with negligible systemic absorption. In certain embodiments, Compound 1 is delivered to the lining of the sigmoid colon with negligible systemic absorption. In certain embodiments, Compound 1 is delivered to the lining of the rectum with negligible systemic absorption. In certain embodiments, Compound 1 is delivered to the lining of the duodenum with negligible systemic absorption. In certain embodiments, Compound 1 is delivered to the lining of the jejunum with negligible systemic absorption. In certain embodiments, Compound 1 is delivered to the lining of the ileum with negligible systemic absorption. In certain embodiments, at most 40%, 30%, 25%, 20%, 15%, 10%, 9%, 8%, 7%, 6%, 5%, 4%, 3%, 2%, 1%, 0.5%, 0.1%, 0.05%, or 0.01% of the orally administered Compound 1 by weight is detectable in the serum.

In certain embodiments, the serum levels of Compound 1 in a patient are measured after oral administration of Compound 1, or a pharmaceutically acceptable salt, tautomer, solvate, or hydrate thereof. In certain embodiments, the serum levels of Compound 1 in the patient are negligible at a time after oral administration of Compound 1. In certain embodiments, the serum levels of Compound 1 are measured immediately, 15 minutes, 30 minutes, 1 hour, 2 hours, 4 hours, 6 hours, 8 hours, 12 hours, 16 hours, 24 hours, 2 days, 3 days, 4 days 5 days, 6 days, 1 week, or 2 weeks following oral administration of Compound 1. In certain embodiments, the serum levels of Compound 1 in the patient are negligible immediately, 15 minutes, 30 minutes, 1 hour, 2 hours, 4 hours, 6 hours, 8 hours, 12 hours, 16 hours, 24 hours, 2 days, 3 days, 4 days 5 days, 6 days, 1 week, or 2 weeks following oral administration of Compound 1. In certain embodiments, the amount of systemic absorption of Compound 1 is negligible. In certain embodiments, at most 40%, 30%, 25%, 20%, 15%, 10%, 9%, 8%, 7%, 6%, 5%, 4%, 3%, 2%, 1%, 0.5%, 0.1%, 0.05%, or 0.01% of the orally administered Compound 1 by weight is detectable in the serum.

### Patient Populations

In certain embodiments, a patient being treated with a method provided herein, e.g., a method for treating or preventing an inflammatory bowel disease comprising administration to a patient having an inflammatory bowel disease a composition comprising an effective amount of Compound 1, or a pharmaceutically acceptable salt, tautomer, solvate, or hydrate thereof, has undergone prior treatment for the inflammatory bowel disease. In certain embodiments, the inflammatory bowel disease is ulcerative colitis. In certain embodiments, the inflammatory bowel disease is Crohn's disease. In specific embodiments, the patient is treated by orally administering Compound 1.

In certain embodiments, a patient being treated with a method provided herein, e.g., a method comprising administration to a patient having an inflammatory bowel disease a composition comprising an effective amount of Compound 1, or a pharmaceutically acceptable salt, tautomer, solvate, or hydrate thereof, is currently being treated with an immunosuppressive or anti-inflammatory agent or drug. In certain embodiments, the immunosuppressive agent is a glucocorticoid, azathioprine, infliximab, adalimumab, golimumab, methotrexate, natalizumab, ustekinumab, mercaptopurine, dactinomycin, anthracycline, mitomycin C, bleomycin, mithramycin, tacrolimus, cyclosporine, sirolimus, everolimus, an opioid, an interferon, a TNF binding protein, mycophenolate, fingolimod, or myriocin. In certain embodiments, the anti-inflammatory agent is sulfasalazine, an aminosalicylate, a corticosteroid, aspirin, ibuprofen, naproxen, paracetamol, celecoxib, diclofenac, diflunisal, etodolac, indomethacin, ketoprofen, ketorolac, nabumetone, oxaprozin, piroxicam, salsalate, sulindac, or tolmetin. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, is unresponsive to treatment with other therapeutic agents. In certain embodiments, the patient is unresponsive to treatment with an immunosuppressive or anti-inflammatory agent. In certain embodiments, the immunosuppressive agent is a glucocorticoid, azathioprine, infliximab, adalimumab, golimumab, methotrexate, natalizumab, ustekinumab, mercaptopurine, dactinomycin, anthracycline, mitomycin C, bleomycin, mithramycin, tacrolimus, cyclosporine, sirolimus, everolimus, an opioid, an interferon, a TNF binding protein, mycophenolate, fingolimod, or myriocin. In certain embodiments, the anti-inflammatory agent is sulfasalazine, an aminosalicylate, a corticosteroid, aspirin, ibuprofen, naproxen, paracetamol, celecoxib, diclofenac, diflunisal, etodolac, indomethacin, ketoprofen, ketorolac, nabumetone, oxaprozin, piroxicam, salsalate, sulindac, or tolmetin. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, is hyporesponsive to treatment with other therapeutic agents. In certain embodiments, the patient is hyporesponsive to treatment with an immunosuppressive or anti-inflammatory agent. In certain embodiments, the immunosuppressive agent is a glucocorticoid, azathioprine, infliximab, adalimumab, golimumab, methotrexate, natalizumab, ustekinumab, mercaptopurine, dactinomycin, anthracycline, mitomycin C, bleomycin, mithramycin, tacrolimus, cyclosporine, sirolimus, everolimus, an opioid, an interferon, a TNF binding protein, mycophenolate, fingolimod, or myriocin. In certain embodiments, the anti-inflammatory agent is sulfasalazine, an aminosalicylate, a corticosteroid, aspirin, ibuprofen, naproxen, paracetamol, celecoxib, diclofenac, diflunisal, etodolac, indomethacin, ketoprofen, ketorolac, nabumetone, oxaprozin, piroxicam, salsalate, sulindac, or tolmetin. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate or hydrate thereof, has a cardiovascular disease. In certain embodiments, the cardiovascular disease is coronary heart disease, rheumatic heart disease, congenital heart disease, peripheral arterial disease, deep venous thrombosis, pulmonary embolism, stroke, hypertensive heart disease, cardiomyopathy, heart arrhythmia, vascular heart disease, carditis, aortic aneurysm, acute heart failure, congestive heart failure, atherosclerosis, restenosis, or vascular stenosis. In certain embodiments, the coronary heart disease is angina or myocoardial infarcation (a “heart attack”). In certain embodiments, the patient is greater than 35, 40, 45, 50, 55, 60, 65, or 70 years old. In certain embodiments, the patient has previously experienced a cardiovascular event. In certain embodiments, the patient has experienced a cardiovascular event in the past week, 2 weeks, 1 month, 3 months, 6 months, 1 year, 2 years, 5 years, or 10 years. In certain embodiments, the cardiovascular event is a heart attack, cardiac arrest, heart failure, stroke, or venous thromboembolism. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, is at risk of developing a cardiovascular disease. In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has or is at risk of developing a cardiovascular disease. In certain embodiments, the cardiovascular disease is coronary heart disease, rheumatic heart disease, congenital heart disease, peripheral arterial disease, deep venous thrombosis, pulmonary embolism, stroke, hypertensive heart disease, cardiomyopathy, heart arrhythmia, vascular heart disease, carditis, aortic aneurysms, acute heart failure, congestive heart failure, atherosclerosis, restenosis, or vascular stenosis. In certain embodiments, the coronary heart disease is angina or myocoardial infarcation (a “heart attack”). In certain embodiments, the patient has a condition that can increase the risk for heart disease. In certain embodiments, the condition is high blood pressure, high cholesterol, obesity, or diabetes. In certain embodiments, the patient's systolic blood pressure is greater than 110, 115, 120, 125, 130, 135, 140, 145, 150, 155, 160, 165, 170, 175, or 180 mm Hg. In certain embodiments, the patient's diastolic blood pressure is greater than 70, 75, 80, 85, 90, 95, 100, 105, or 110 mm Hg. In certain embodiments, the patient has a diet high in saturated fats, trans fats, or cholesterol. In certain embodiments, the patients cholesterol levels are greater than 180, 185, 190, 195, 200, 205, 210, 215, 220, 225, 230, 235, or 240 mg/dL. In certain embodiments, the patient is a smoker. In certain embodiments, the patient has a higher chance of developing a cardiovascular disease than the population at large. In certain embodiments, the patient has a 5-10%, 10-15%, 15-20%, 20-25%, 25-30%, 30-35%, 45-40%, 40-45%, 45-50%, 50-55%, 55-60%, 60-65%, 65-70%, 70-75%, 75-80%, 80-85%, 85-90%, or a greater than 90% increased risk of developing a cardiovascular disease. In certain embodiments, the patient has one or more family members who have or have had a cardiovascular disease. In certain embodiments, the patient is greater than 35, 40, 45, 50, 55, 60, 65, or 70 years old. In certain embodiments, the patient has previously experienced a cardiovascular event. In certain embodiments, the patient has experienced a cardiovascular event in the past week, 2 weeks, 1 month, 3 months, 6 months, 1 year, 2 years, 5 years, or 10 years. In certain embodiments, the cardiovascular event is a heart attack, cardiac arrest, heart failure, stroke, or venous thromboembolism. In certain embodiments, the patient has vascular calcification. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has diabetes. In certain embodiments, the diabetes is Type 1 diabetes. In certain embodiments, the diabetes is Type 2 diabetes. In certain embodiments, the diabetes is gestational diabetes. In certain embodiments, the diabetes is prediabetes. In certain embodiments, the diabetes is maturity onset diabetes of the young. In certain embodiments, the diabetes is latent autoimmune diabetes of adults. In certain embodiments, the diabetes is congenital diabetes. In certain embodiments, the diabetes is steroid diabetes. In certain embodiments, the diabetes is monogenic diabetes. In certain embodiments, the patient's random (non-fasting) blood glucose levels are at least 140, 145, 150, 155, 160, 165, 170, 175, 180, 185, 190, 195, or 200 mg/dL. In certain embodiments, the patient's fasting blood glucose levels are at least 100, 105, 110, 115, 120, 125, 130, 135, 140, 145, or 150 mg/dL. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, is at risk of developing diabetes. In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has or is at risk of developing diabetes. In certain embodiments, the diabetes is Type 1 diabetes. In certain embodiments, the diabetes is Type 2 diabetes. In certain embodiments, the diabetes is gestational diabetes. In certain embodiments, the diabetes is prediabetes. In certain embodiments, the diabetes is maturity onset diabetes of the young. In certain embodiments, the diabetes is latent autoimmune diabetes of adults. In certain embodiments, the diabetes is congenital diabetes. In certain embodiments, the diabetes is steroid diabetes. In certain embodiments, the diabetes is monogenic diabetes. In certain embodiments, the patient's random (non-fasting) blood glucose levels are at least 140, 145, 150, 155, 160, 165, 170, 175, 180, 185, 190, 195, or 200 mg/dL. In certain embodiments, the patient's fasting blood glucose levels are at least 100, 105, 110, 115, 120, 125, 130, 135, 140, 145, or 150 mg/dL. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has a disease or disorder in which angiogenesis is contraindicated. In certain embodiments, the disease or disorder is cancer. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, is at risk of developing a disease or disorder in which angiogenesis is contraindicated. In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has or is at risk of developing a disease or disorder in which angiogenesis is contraindicated. In certain embodiments, the disease or disorder is cancer. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has a disease or disorder in which erythropoiesis is contraindicated. In certain embodiments, the disease or disorder is uncontrolled hypertension. In certain embodiments, the disease or disorder is pure red cell aplasia. In specific embodiments, the patient is treated by orally administering Compound 1.

In certain embodiments, a patient being treated with a method provided herein, e.g., a method comprising administration to a patient a composition comprising an effective amount of Compound 1, or a pharmaceutically acceptable salt, tautomer, solvate, or hydrate thereof, is at risk of developing a disease or disorder in which erythropoiesis is contraindicated. In certain embodiments, a patient being treated with a method provided herein, e.g., a method comprising administration to a patient a composition comprising an effective amount of Compound 1, or a pharmaceutically acceptable salt, tautomer, solvate, or hydrate thereof, has or is at risk of developing a disease or disorder in which erythropoiesis is contraindicated. In certain embodiments, the disease or disorder is uncontrolled hypertension. In certain embodiments, the disease or disorder is pure red cell aplasia. In specific embodiments, the patient is treated by orally administering Compound 1.

### Formulations and Routes of Administration

Compound 1, or pharmaceutically acceptable salts, tautomers, solvates, or hydrates thereof, can be delivered to a patient using oral administration.

For any mode of administration, the actual amount of Compound 1 delivered will depend, in part, on such factors as the disorder being treated, the desired therapeutic dose, and other factors that will be apparent to those of skill in the art. The actual amounts delivered and dosing schedules can be readily determined by those of skill without undue experimentation.

Compound 1, its pharmaceutically acceptable salts, tautomers, solvates, and/or hydrates thereof, may be administered in combination with other compounds, and/or in combination with other therapeutic agents. Compound 1 may be administered in the form of a pharmaceutical composition, wherein Compound 1 is in admixture with one or more pharmaceutically acceptable carriers, excipients, or diluents. Pharmaceutical compositions for use in accordance with the methods described herein may be formulated in conventional manner using one or more physiologically acceptable carriers comprising excipients and auxiliaries which facilitate processing of Compound 1 into preparations, which can be used pharmaceutically.

Pharmaceutical compositions may be used in the preparation of individual, single unit dosage forms. Pharmaceutical compositions and dosage forms provided herein comprise Compound 1, or a pharmaceutically acceptable salt, tautomer, solvate, or hydrate thereof. Pharmaceutical compositions and dosage forms can further comprise one or more excipients.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets.

In certain embodiments, Compound 1 is formulated in an oral formulation. In specific embodiments such oral formulations comprising Compound 1 do not comprise enteric coating. An oral formulation can be a tablet or a capsule. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide. In certain embodiments, the carrier is a carrier that is suitable for oral formulation. In certain embodiments, the excipient is an excipient that is suitable for oral formulation. In certain embodiments, the diluent is a diluent that is suitable for oral formulation.

Oral tablets may further include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating. In certain embodiments, the tablets are not coated with an enteric coating.

Capsules for oral administration may further include hard and soft gelatin capsules. To prepare hard gelatin capsules, compounds of the invention may be mixed with a solid, semi-solid, or liquid diluent. Soft gelatin capsules may be prepared by mixing Compound 1 of the invention with water, an oil such as peanut oil or olive oil, liquid paraffin, a mixture of mono and di-glycerides of short chain fatty acids, polyethylene glycol 400, or propylene glycol. Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds, compositions, methods, and uses disclosed herein are not to be limited in scope by the specific embodiments described herein. Indeed, various modifications of the compounds, compositions, methods, and uses in addition to those described will become apparent to those skilled in the art from the foregoing description and accompanying figures. Such modifications are intended to fall within the scope of the appended claims.

### EXAMPLES

### Example 1

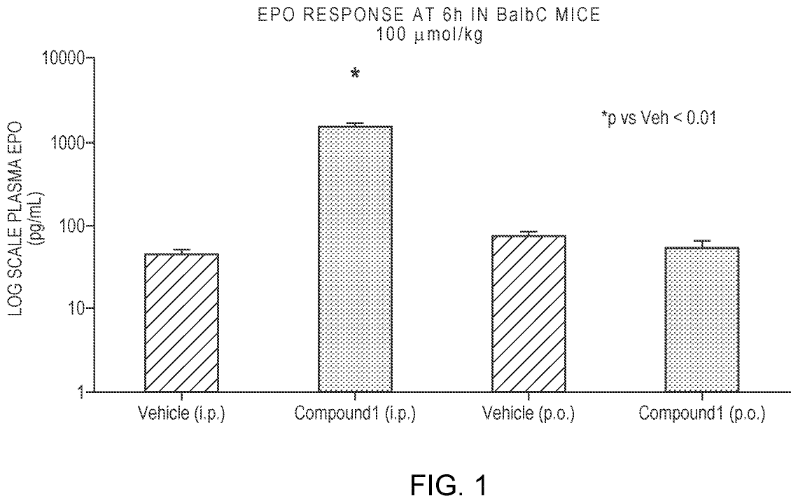

The effect of both oral (p.o.) and intraperitoneal (i.p.) doses of Compound 1 on systemic EPO in male Balb/c mice was investigated, along with plasma drug concentrations at 1 h and 6 h post dosing. Systemic EPO was measured 6 h post dosing of Compound 1 using a commercially-available MSD assay system, correlated to standard curves. Plasma drug concentrations were quantitated by LC MS/MS. Parenteral (100 umol/kg i.p.) dosing of Compound 1 resulted in blood levels of 42.2 uM and 5.1 uM at 1 h and 6 h, respectively, producing an EPO stimulation of 34-fold above vehicle control. Oral dosing (100 umol/kg p.o.) of Compound 1 resulted in blood levels of 0.10 uM and 0.02 uM at lh and 6h, respectively, producing no EPO stimulation. FIG. 1 depicts the EPO response to oral (p.o.) and intraperitoneal (i.p.) doses of Compound 1 at 6 h in BalbC mice. This data demonstrates that, when Compound 1 is dosed IP at 100 uM/Kg, plasma levels are sufficient to induce increases in EPO. Because the compound is poorly absorbed following p.o. dosing, plasma levels following a 100 uM/Kg dose are low and the Compound 1 does not induce increases in EPO.

### Example 2

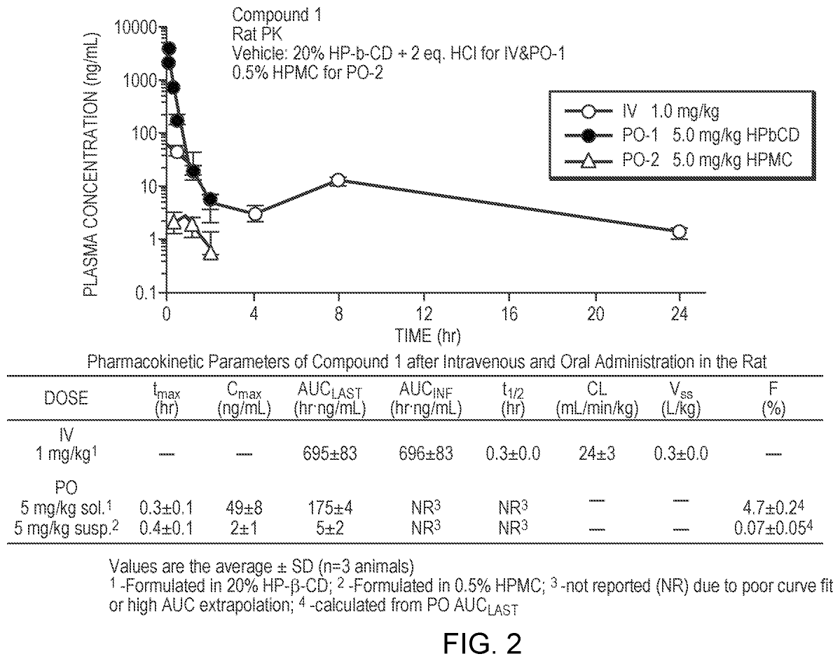

Separate cohorts (n=3) of non-fasted male Sprague Dawley rats were administered Compound 1 at a dose of 1 mg/kg intravenously (IV) via tail vein, 5 mg/kg orally as a solution, or 5 mg/kg orally as a suspension. Blood samples were collected at 0.033 (IV only), 0.083 (IV only), 0.25, 0.5, 1, 2, 4, 8, and 24 hrs post dose after administration. Blood was harvested via the jugular vein catheter. Blood samples were collected into tubes containing K2EDTA and placed on wet ice. The plasma fraction was separated by centrifugation and frozen at −20 to −80 ° C. Concentrations of Compound 1 in plasma were determined using a qualified liquid chromatography-triple quadrupole mass spectrometry. FIG. 2 depicts the plasma concentration of Compound 1 in healthy male SD rats over 24 hours. This example shows that Compound 1 has low bioavailability following p.o. dosing in rats.

### Example 3

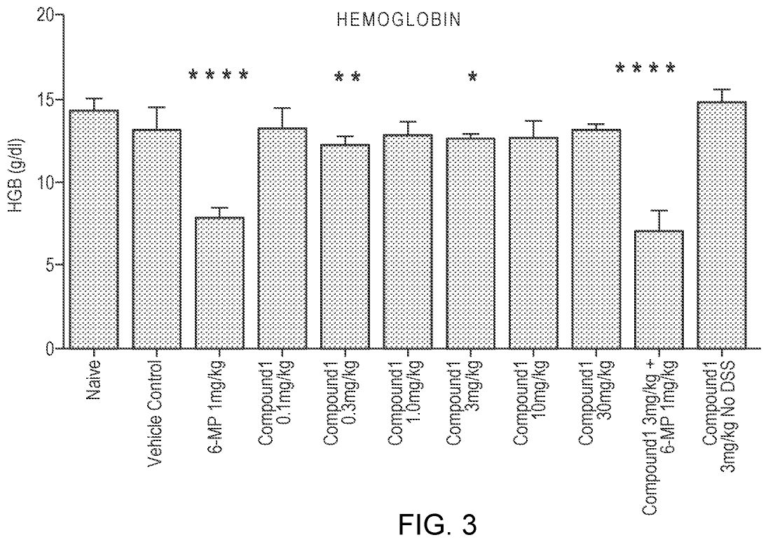

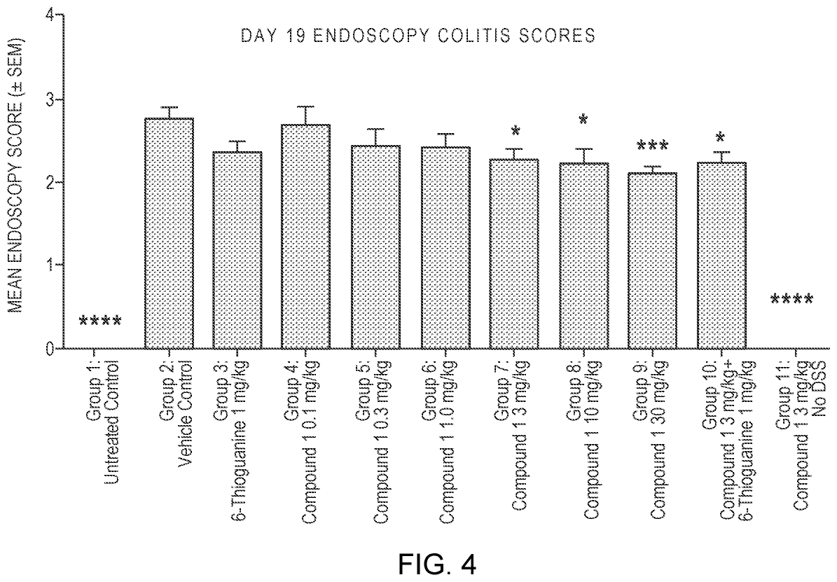

Colitis was induced in 121 mice by exposure to 3% DSS-treated drinking water from Day 0 to Day 5. Animals were dosed via oral gavage (p.o.) with Compound 1, 6-thioguanine, or vehicle control once a day (q.d.) from Day 6 to 19. Video endoscopy was performed on days 10, 14, and 19. At these time points, colitis severity was assessed in all animals using video endoscopy, where images were taken and colitis severity scored by a blinded observer. During each endoscopy procedure, stool consistency was recorded using a 0-4 scale and an image from each animal was captured at the most severe region of disease that was identified. Following endoscopy on Day 19, all animals for the efficacy component were euthanized with CO₂ inhalation. Blood was collected via cardiac puncture and placed in K2EDTA tubes for preparation of plasma. A small whole blood sample was retained for the assessment of hemoglobin and hematocrit while the remaining sample was processed to plasma, then split into two samples and snap frozen. A complete blood count, including hematocrit and hemoglobin levels, was assessed using an automated PCE-90 Vet hematology unit (HTI). FIG. 3 depicts the serum hemoglobin concentration after 14 days of oral dosing Compound 1 in mice with dextran sulfate sodium (DSS)-induced colitis. FIG. 4 depicts Endoscopy Colitis Scores in mice with dextran sulfate sodium (DSS)-induced colitis dosed via oral gavage with Compound 1, 6-thioguanine, or vehicle control once a day after 19 days. This example illustrates that Compound 1 is effective at ameliorating DSS induced colitis as assessed by Endoscopic Colitis Scores.

### Example 4

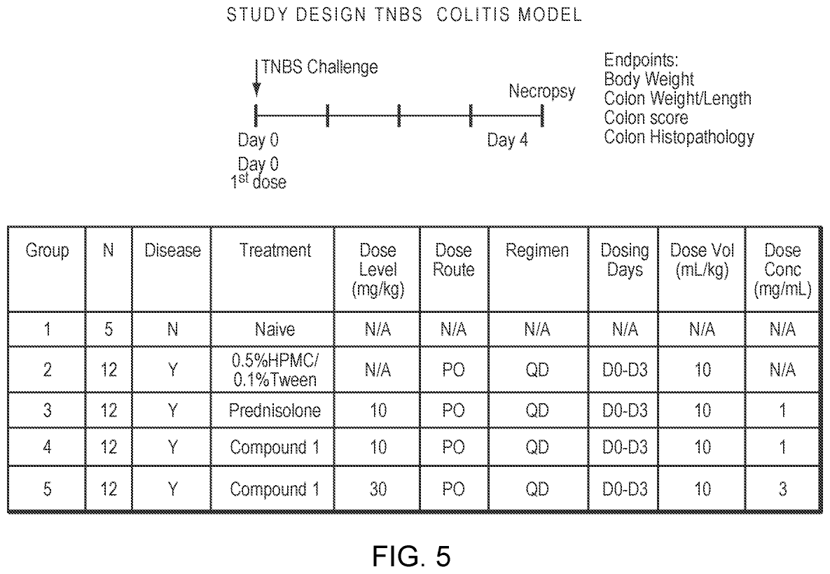

A study was conducted to determine the effects of Compound 1 compared to positive control prednisolone dosed by the oral (p.o.) route in trinitrobenzene sulfonic acid (TNBS)-induced colitis in male SJL mice (FIG. 5). The mice were divided into 5 groups. The mice were fasted for 12 to 16 hours prior to TNBS challenge. On day 0 of the study, mice in groups 2 through 5 were infused intrarectally (IR) with 50 μL TNBS solution (1.5 mg in 50% EtOH/PBS per mouse) to induce colitis. The mice in group 2 were dosed (PO) daily (QD) on days 0 to 3 with vehicle (0.5% HPMC, 0.1% Tween 80 in water). The mice in group 4 were dosed p.o. daily on days 0 to 3 with Compound 1 at 10 mpk, and the mice in group 5 were dosed p.o. daily on days 0 to 3 with Compound 1 at 30 mpk. Prednisone (group 3) was used as a positive control and was dosed p.o., QD on days 0 to 3. Group 1 served as naïve controls. On study day 4, the mice were anesthetized with Isoflurane, bled to exsanguination, and then euthanized for necropsy and tissue collection. Efficacy evaluation was based on animal body weight measurements, colon weights, colon lengths, colon weight per length ratios, histopathology of proximal and distal colons—evaluated as proximal only, distal only, or full colon (proximal and distal).

### Histopathology

Longitudinal sections of proximal and distal colon were stained with hematoxylin and eosin (H&E). Sections were analyzed using the criteria below and were scored for inflammation, gland loss, and mucosal necrosis.

Inflammation Scoring Criteria

| Score | Observations |
|---|---|
| 0 = | No inflammation |
| 0.5 = | Very minimal focal infiltrates in mucosa only, affects <2% of the total |
| | colon |
| 1 = | Minimal focal or multifocal infiltrates in mucosa and/or submucosa, affects |
| | 2-10% of the total colon in areas of necrosis, or is minimal and diffuse |
| | throughout the mucosa if there is no necrosis |
| 2 = | Mild focal or multifocal infiltrates affecting mucosa, submucosa, outer |
| | muscle layers and serosa, affects 11-25% of the total colon in area of |
| | necrosis and is minimal to mild throughout the rest of the mucosa, or mild |
| | and diffuse throughout the mucosa if there is no necrosis |
| 3 = | Moderate focal or multifocal infiltrates affecting mucosa, submucosa, outer |
| | muscle layers and serosa, affects 26-50% of the total colon in area of |
| | necrosis and is mild throughout the rest of the mucosa, or moderate and |
| | diffuise throughout the mucosa if there is no necrosis |
| 4 = | Marked multifocal to diffuse infiltrates affecting mucosa, submucosa, outer |
| | muscle layers and serosa, affects 51-75% of the total colon in area of |
| | necrosis and is mild to moderate throughout the rest of the mucosa |
| | Severe multifocal to diffuse infiltrates affecting mucosa, submucosa, outer |
| | muscle layers and serosa, affects >75% of the total colon in area of necrosis |
| | and is moderate to marked throughout the rest of the mucosa |

Gland Loss Scoring Criteria

| Score | Observation |
|---|---|
| 0 = | No loss |
| 0.5 = | Very minimal, affects <2% of the toal colon |
| 1 = | Minimal, focal or multifocal, affects 2-10% of the colon |
| 2 = | Mild, focal or multifocal, affects 11-25% of the colon |
| 3 = | Moderate, focal or multifocal, affects 26-50% of the colon |
| 4 = | Marked, multifocal to diffuse, affects 51-75% of the total colon |
| 5 = | Severe, multifocal to diffuse, affects >75% of the total colon |

Erosion/Necrosis Scoring Criteria

| Score | Observations |
|---|---|
| 0 = | No necrosis |
| 0.5 = | Very minimal, focal, affects <2% of the toal colon |
| 1 = | Minimal, focal or multifocal, affects 2-10% of the colon |
| 2 = | Mild, focal or multifocal, affects 11-25% of the colon |
| 3 = | Moderate, focal or multifocal, affects 26-50% of the colon |
| 4 = | Marked, multifocal to diffuse, affects 51-75% of the total colon |
| 5 = | Severe, affects >75% of the total colon |

Hyperplasia Scoring Criteria

| Score | Observations |
|---|---|
| 0 = | ≤200 μm = normal |
| 0.5 = | 201-350 μm = very minimal |
| 1 = | 251-350 μm = minimal |
| 2 = | 351-450 μm = mild |
| 3 = | 451-550 μm = moderate |
| 4 = | 551-650 μm = marked |
| 5 = | ≥651 μm = severe |

The histopathology sum score was calculated as a sum of inflammation, gland loss, erosion/necrosis (derived from the measure of total length vs. length of mucosal necrosis, i.e., percent), and hyperplasia scores.

Results

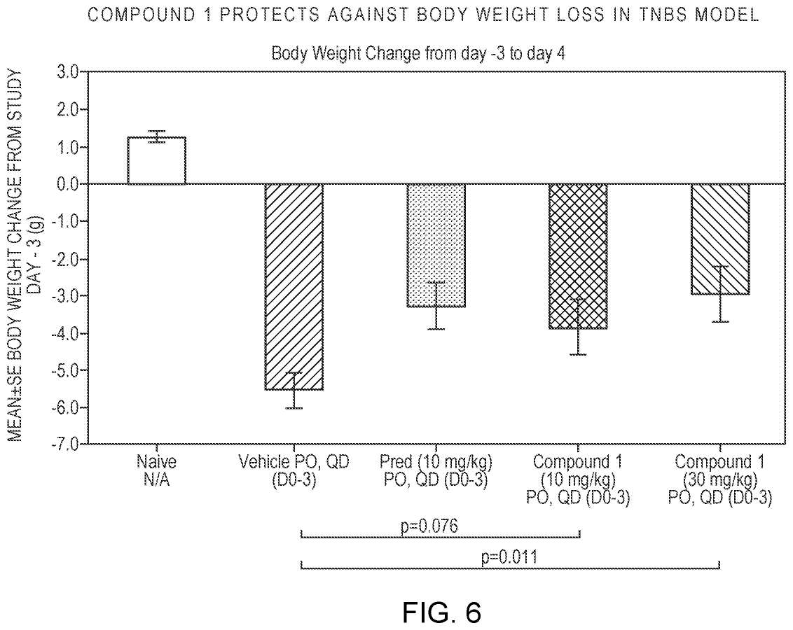

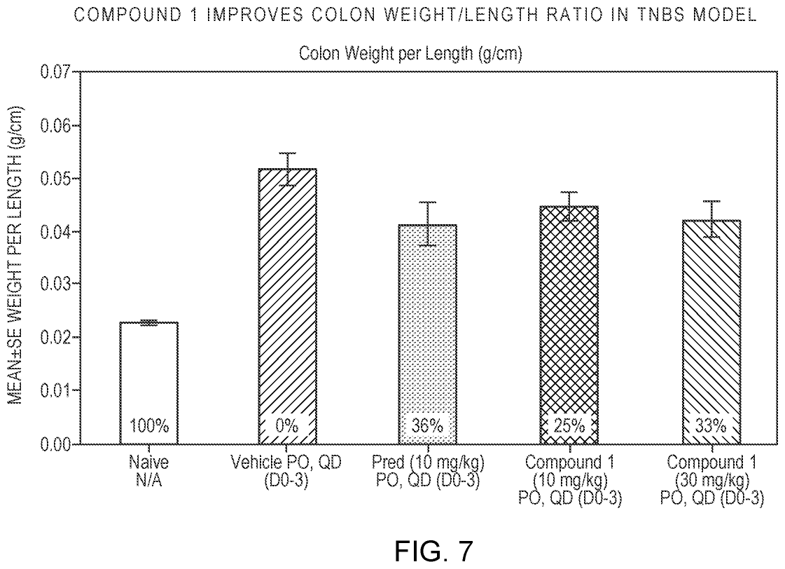

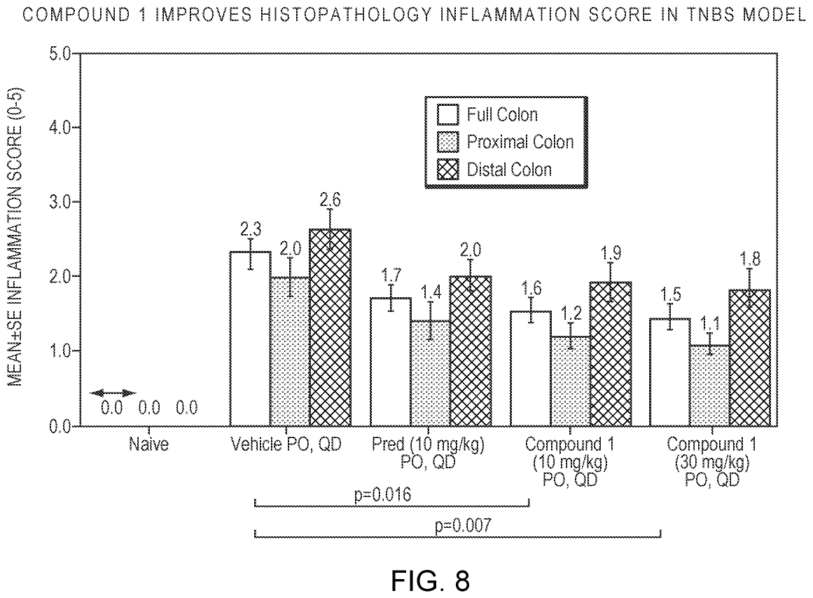

Oral treatment with Compound 1 dosed at 10 and 30 mg/kg showed beneficial effects on TNBS-induced colitis in male SJL mice as determined by statistically significant inhibition of body weight loss (FIG. 6) along with decreased colon weight/length ratios (FIG. 7) and improvements in colon histopathology (FIGS. 8 & 9). Treatment with Compound 1 at 10 or 30 mpk resulted in significant beneficial effects on full-colon and proximal-colon histopathology.

### Example 5

Intravenous and oral (IV/p.o.) pharmacokinetic (PK) studies in rat, dog and monkey were carried out to facilitate the projection of human PK for Compound 1.

In the rat studies, Compound 1 was dosed both as a suspension (in 0.5% HPMC) and as a solution in 20% HPPCD. Experiments in the dog and monkey were dosed as a suspension in 0.5% HPMC.

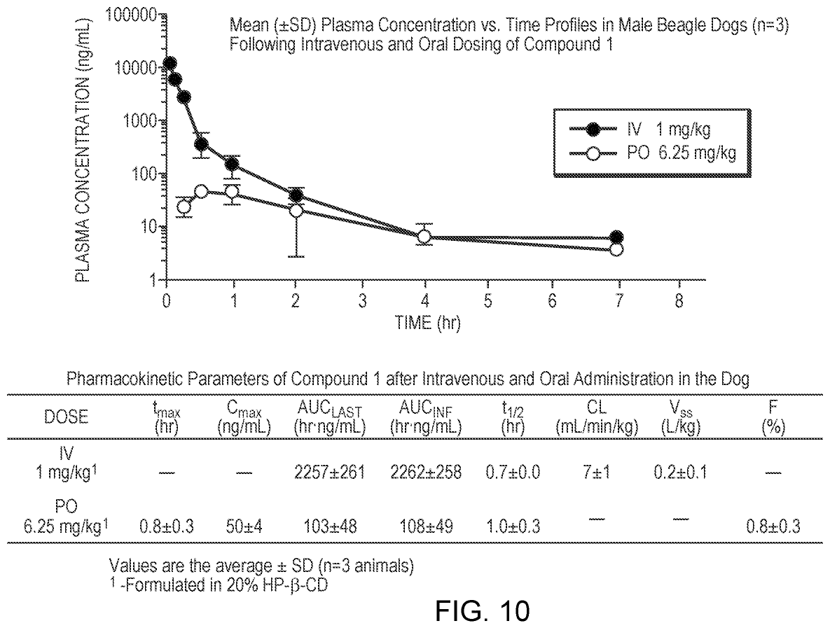

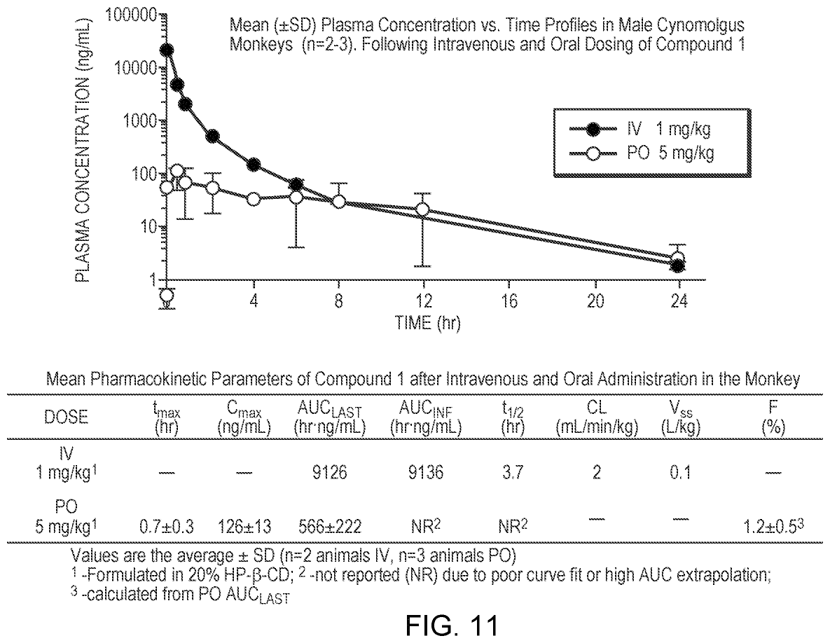

In rat, dog and monkey, the pharmacokinetic properties of Compound 1 are characterized by low oral bioavailability (≤5% in rat, dog, monkey) and short terminal t_{1/2}. Based on these results, estimated fₐ*f_{g} (fₐ: fraction absorbed; f_{g}: fraction that escapes gut metabolism) in rat, dog and monkey is low (≤0.12). (FIGS. 2 and 10-11)

In Caco-2 cell monolayers, Compound 1 had low apical-to-basolateral apparent permeability Pₐₚₚ value of <0.2×10⁻⁶ cm/s when tested at 10 μM.

Taken together, these data suggest that the fraction absorbed, oral bioavailability, and plasma exposures of Compound 1 would be low in humans.

What is claimed:

1. A method for treating or preventing inflammatory bowel disease in a patient in need thereof, comprising administering orally to a patient in need thereof an effective amount of Compound 1: 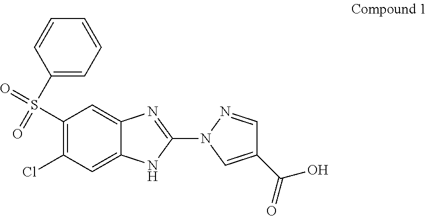 [SMILES: O=C(O)c1cnn(-c2nc3cc(S(=O)(=O)c4ccccc4)c(Cl)cc3[nH]2)c1]
or a pharmaceutically acceptable salt, tautomer, solvate or hydrate thereof.

2. The method of claim 1, wherein the inflammatory bowel disease is ulcerative colitis.

3. The method of claim 2, wherein the ulcerative colitis is mild to moderate ulcerative colitis.

4. The method of claim 2, wherein the ulcerative colitis is moderate to severe ulcerative colitis.

5. The method of claim 2, wherein the ulcerative colitis is ulcerative proctitis.

6. The method of claim 2, wherein the ulcerative colitis is proctosigmoiditis.

7. The method of claim 2, wherein the ulcerative colitis is left-sided colitis.

8. The method of claim 2, wherein the ulcerative colitis is pan-ulcerative colitis.

9. The method of claim 2, wherein the patient has undergone prior treatment for ulcerative colitis.

10. The method of claim 2, wherein the patient is currently being treated with an anti-inflammatory drug.

11. The method of claim 1, wherein the inflammatory bowel disease is Crohn's disease.

12. The method of claim 11, wherein the Crohn's disease is ileocolitis.

13. The method of claim 11, wherein the Crohn's disease is ileitis.

14. The method of claim 11, wherein the Crohn's disease is gastroduodenal Crohn's disease.

15. The method of claim 11, wherein the Crohn's disease is jejunoileitis.

16. The method of claim 11, wherein the Crohn's disease is Crohn's (granulomatous) colitis.

17. The method of claim 11, wherein the patient has a Crohn's Disease Activity Index (“CDAI”) score greater than or equal to 150 and less than or equal to 220.

18. The method of claim 11, wherein the patient has a CDAI score greater than or equal to 220 and less than or equal to 300.

19. The method of claim 11, wherein the patient has a CDAI score greater than

300.

20. The method of claim 1, wherein the composition is administered daily.

21. The method of claim 1, wherein the oral bioavailability of Compound 1 is less than 10%.

22. The method of claim 1, wherein the compound is delivered to the intestinal epithelium of the patient with negligible systemic absorption.

23. The method of claim 1, wherein the compound is delivered to the colon of the patient with negligible systemic absorption.

24. The method of claim 1, wherein the compound is delivered to the lining of the descending colon of the patient with negligible systemic absorption.

25. The method of claim 1, wherein serum levels of the compound in the patient are negligible at a time after administration of the compound.

26. The method of claim 1, wherein the change in hemoglobin levels in the patient are negligible six hours after administration of the compound.

27. The method of claim 1, wherein the change in EPO levels in the patient are negligible six hours after oral administration of the compound.

28. The method of claim 1, wherein the change in red blood cell levels in the patient are negligible six hours after oral administration of the compound.

29. The method of claim 1, wherein the patient is at risk of developing a cardiovascular disease.

30. The method of claim 29, wherein the patient has experienced a cardiovascular event in the last two years.

31. The method of claim 30, wherein the cardiovascular event is a heart attack or stroke.

32. The method of claim 1, wherein the patient has a disorder in which angiogenesis is contraindicated.

33. The method of claim 1, wherein the patient has a disorder in which erythropoiesis is contraindicated.

34. The method of claim 1, wherein Compound 1 is administered as an oral formulation.

35. The method of claim 34, wherein the oral formulation is a tablet or capsule.

36. The method of claim 34, wherein the oral formulation does not comprise an enteric coating.

37. A method of healing the epithelial layer of the descending colon in a patient in need thereof by administering orally an effective amount of Compound 1: 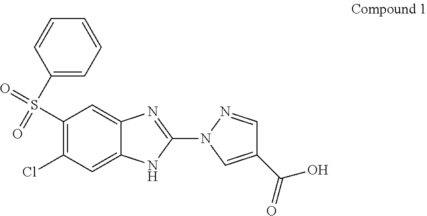 [SMILES: O=C(O)c1cnn(-c2nc3cc(S(=O)(=O)c4ccccc4)c(Cl)cc3[nH]2)c1]
or a pharmaceutically acceptable salt, tautomer, solvate or hydrate thereof.

38. The method of claim 37, wherein the composition is administered daily.

39. The method of claim 37, wherein the bioavailability of Compound 1 is less than 10%.

40. The method of claim 37, wherein the compound is delivered to the intestinal epithelium of the patient with negligible systemic absorption.

41. The method of claim 37, wherein the compound is delivered to the colon of the patient with negligible systemic absorption.

42. The method of claim 37, wherein the compound is delivered to the lining of the descending colon of the patient with negligible systemic absorption.

43. The method of claim 37, wherein serum levels of the compound in the patient are negligible at a time after administration of the compound.

44. The method of claim 37, wherein the change in hemoglobin levels in the patient are negligible six hours after administration of the compound.

45. The method of claim 37, wherein the change in EPO levels in the patient are negligible six hours after oral administration of the compound.

46. The method of claim 37, wherein the change in red blood cell levels in the patient are negligible six hours after oral administration of the compound.

47. The method of claim 37, wherein the patient is at risk of developing a cardiovascular disease.

48. The method of claim 37, wherein the patient has a disorder in which angiogenesis is contraindicated.

49. The method of claim 37, wherein the patient has a disorder in which erythropoiesis is contraindicated.

50. The method of claim 37, wherein the composition is formulated as an oral formulation.

51. The method of claim 50, wherein the oral formulation is a tablet or capsule.

52. The method of claim 50, wherein the oral formulation does not comprise an enteric coating.

53. A method for maintaining remission of ulcerative colitis in a patient, comprising administering orally to a patient an effective amount of Compound 1: 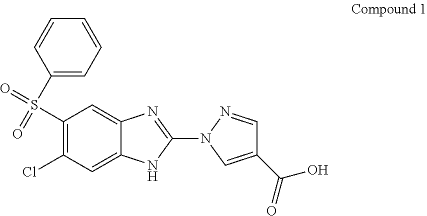 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof after remission has been achieved.

54. The method of claim 53, wherein the ulcerative colitis is mild to moderate ulcerative colitis.

55. The method of claim 53, wherein the ulcerative colitis is moderate to severe ulcerative colitis.

56. The method of claim 53, wherein the remission has been achieved by treatment with immunosuppressive agents, inflammatory agents, or surgery.

57. A method for inducing remission of ulcerative colitis or Crohn's disease in a patient, comprising administering orally to a patient an effective amount of Compound 1: 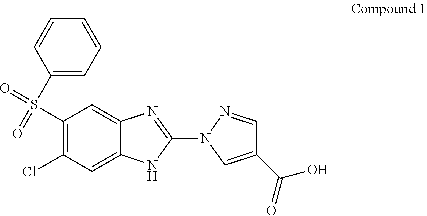 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof after remission has been achieved.

58. The method of claim 57, wherein the ulcerative colitis is mild to moderate ulcerative colitis.

59. The method of claim 57, wherein the ulcerative colitis is moderate to severe ulcerative colitis.

60. A method for inducing mucosal healing in a patient in need thereof, comprising administering orally to a patient an effective amount of Compound 1: 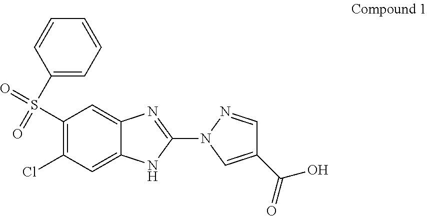 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof.

61. A method for treating or preventing ulcerative colitis in a male patient, comprising administering orally to a male patient an effective amount of Compound 1: 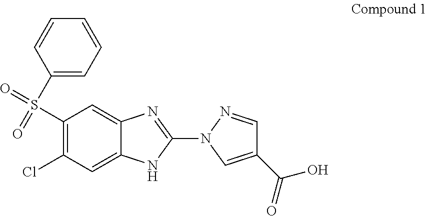 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof, wherein the hemoglobin level in the male patient is 13.5 to 17.5 grams per deciliter.

62. The method of claim 61, wherein the ulcerative colitis is mild to moderate ulcerative colitis.

63. The method of claim 61, wherein the ulcerative colitis is moderate to severe ulcerative colitis.

64. A method for treating or preventing ulcerative colitis in a female patient, comprising administering orally to a female patient an effective amount of Compound 1: 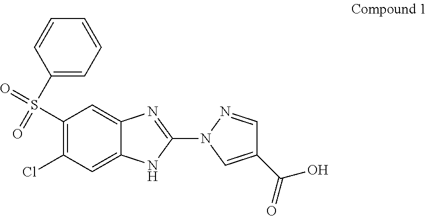 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof, wherein the hemoglobin level in the female patient is 12.0 to 15.5 grams per deciliter.

65. The method of claim 64, wherein the ulcerative colitis is mild to moderate ulcerative colitis.

66. The method of claim 64, wherein the ulcerative colitis is moderate to severe ulcerative colitis.

67. A method for treating or preventing esophagitis in a patient in need thereof, comprising administering to a patient in need thereof an effective amount of Compound 1: 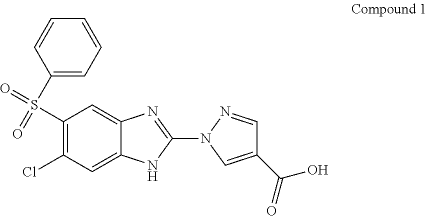 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof.

68. The method of claim 67, wherein the esophagitis is eosinophilic esophagitis.

69. A method for treating or preventing gastritis in a patient in need thereof, comprising administering to a patient in need thereof an effective amount of Compound 1: 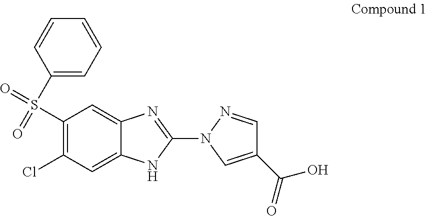 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof.

70. A method for treating or preventing pouchitis in a patient in need thereof, comprising administering to a patient in need thereof an effective amount of Compound 1: 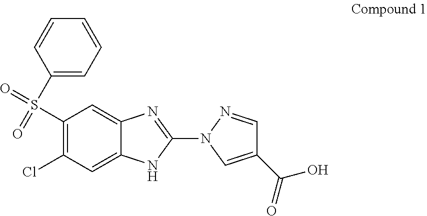 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof.

71. A method for treating or preventing mucositis in a patient in need thereof, comprising administering to a patient in need thereof an effective amount of Compound 1: 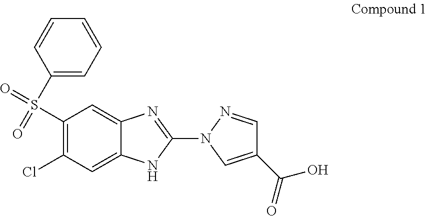 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof.

72. An oral formulation comprising
(i) Compound 1: 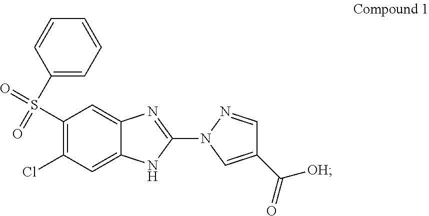 [SMILES: O=C(O)c1cnn(-c2nc3cc(S(=O)(=O)c4ccccc4)c(Cl)cc3[nH]2)c1]
or a pharmaceutically acceptable salt, tautomer, solvate, or hydrate thereof, and
(ii) one or more excipients or carriers suitable for oral administration.

73. The oral formulation of claim 72, wherein the oral formulation is a tablet or capsule.

74. The oral formulation of claim 72, wherein the oral formulation does not comprise an enteric coating.
